(19) United States
(12) Patent Application Publication
(10) Pub. No.: US 2011/0295140 A1
(43) Pub. Date: Dec. 1, 2011

(54) Method and Apparatus for Measuring Trace Levels of CO in Human Breath Using Cavity Enhanced, Mid-Infared Absorption Spectroscopy

(75) Inventors: Sohail H. Zaidi, Pennington, NJ (US); Azer Yalin, Fort Collins, CO (US)

(73) Assignee: INTELLISCIENCE RESEARCH LLC, Princeton, NJ (US)

(21) Appl. No.: 12/789,632

(22) Filed: May 28, 2010

(51) Int. Cl. A61B 5/08

(52) U.S. Cl. 600/532

(57) ABSTRACT

A method and apparatus for analyzing trace levels of CO in human breath for the purpose of, among other things, assessing the severity of pulmonary diseases and monitoring the patient's response to a prescribed treatment. The apparatus measures in situ and in real time the CO concentration at sensitivity levels at least as low as parts per billion. A laser of the apparatus has a wavelength in mid-infrared (MIR) spectrum. The optical is constructed and arranged to perform cavity enhanced absorption spectroscopy (CEAS) the breath sample. The cavity includes highly reflective mirrors mounted to each side of the optical cavity to cause the light received from the laser to bounce back and forth within the optical cavity to increase effective path length of the light. A breath intake tube is connected to the optical cavity for collecting a sample of the patient's exhaled breath and transferring it to the optical cavity. A photo detector measures parameters of the light exiting the optical cavity. A controller to operates the system and determines the concentration of CO in the breath sample based on measurements from the photo detector. Appropriate hardware and software display and store the data in real time.

### FIELD OF INVENTION

The present invention relates to a method and apparatus for analyzing trace levels of CO in human breath for the purpose of, among other things, assessing the severity of pulmonary diseases and monitoring the patient's response to a prescribed treatment.

### BACKGROUND OF THE INVENTION

Many lung diseases including asthma, chronic obstructive pulmonary disease (COPD), pre-eclampsia, and cystic fibrosis (CF) involve chronic inflammation and oxidative stress. These conditions cannot be measured directly in routine clinical practice because of the difficulties in monitoring inflammation using invasive techniques such as bronchoscopy and bronchoalveolar lavage. As a result, non-invasive techniques have been developed to indirectly monitor inflammation in the lungs by analyzing exhaled gases and condensates in human breath. While human breath mainly consists of carbon dioxide, it also includes other gases such as nitric oxide (NO) and carbon monoxide (CO) at trace levels. It has been noted that NO and CO in human breath are quantitatively correlated with their respective levels in the blood stream.

The variation in CO concentration in human breath at part per billion (ppb) levels can be used as a supplemental diagnostic parameter for several pulmonary diseases, such as those described above, for assessing disease severity, and monitoring a patient's response to treatment. The variation in CO concentration can also be used for other diagnostic applications including monitoring lung transplant and neonatal intensive care patients. Furthermore, CO concentration monitoring at early stages may assist greatly in preventing further progression of the above-mentioned debilitating and deadly diseases.

There have been many studies correlating a patient's increased or decreased respiratory CO concentration to various ailments. For example, studies have shown that exhaled CO concentrations are significantly increased in non-steroid treated asthmatic patients compared with healthy subjects. Studies have demonstrated that exhaled CO concentration in control groups is less than in stable cystic fibrosis (CF) groups, which, in turn, is less than unstable CF groups. A recent study further demonstrated that at 0-12 h after birth, end-tidal CO (ETCOc) levels were significantly higher in infants with hemolysis, elevated liver enzymes, low platelets syndrome (HELLP) compared to infants from pre-eclamptic mothers without HELLP. Due to current technological limits, the qualitative measurements in these studies has been in the sensitivity range of parts per million (ppm). In order to more accurately detect and monitor these and other conditions, it would be desirable to provide a non-invasive, commercially-available breath analyzing device that can measure CO concentrations in the sensitivity range of parts per billion (ppb).

### SUMMARY OF THE INVENTION

The present invention provides a non-invasive, in situ method and apparatus for analyzing trace levels of CO in human breath for providing a supplemental diagnostic parameter for pulmonary diseases, assessing the severity of the disease, and monitoring the patient's response to a prescribed treatment. The apparatus collects a sample of the air exhaled by a patient and measures its CO concentration at sensitivity levels as low as parts per billion. The apparatus processes and displays the CO concentration in real time on a displaying unit. The displaying unit has both graphical and numerical display options, thereby providing a real-time, in situ visual means for measuring and monitoring CO concentrations in the patient's breath. In a preferred embodiment, the apparatus is compact and installed on a moveable cart for easy transport from one location to another.

In a preferred embodiment, the apparatus collects a sample of the patient's breath and performs cavity enhanced absorption spectroscopy (CEAS) on the sample. The apparatus includes a breath sampling unit into which a patient exhales a breath sample for analysis. An optical cavity is located within the sampling unit. A laser beam is tuned and injected into the optical cavity for performing CEAS on the breath sample. A photodetector measures parameters of the laser beam exiting the optical cavity of the unit. A controller controls operation of the apparatus and calculates the CO concentration within the breath sample based on measurements from the photodetector. The controller includes a processor and a controller-readable storage medium, which stores controller executable instructions that cause the controller to control operation of the system to perform CEAS and display, in real time, the CO concentration within the sample.

In a preferred embodiment, the apparatus performs cavity-enhanced ring-down spectroscopy on the sample. However, various other versions of CEAS can be performed, including integrated cavity output spectroscopy (ICOS), phase-shift cavity ring-down spectroscopy (PS-CRDS), continuous wave cavity enhanced absorption spectrometry (cw-CEAS), noise-immune cavity-enhanced optical-heterodyne molecular spectroscopy (NICE-OHMS), and related techniques that use optical cavities to achieve high detection sensitivity.

The breath sampling unit preferably includes a breath intake tube connecting the optical cavity to a mouthpiece into which the patient exhales. The intake tube includes a flow meter and adjustable valve connected to the controller, which control the volume of breath sample provided to the optical cavity. The breath sampling unit also includes a purge gas inlet and outlet tubes connected to and providing an appropriate atmosphere within the optical cavity. The purge gas inlet and outlet tubes preferably include a flow meter and adjustable valve connected to the controller, which control the pressure and volume of atmospheric purge gas in the optical cavity. Temperature controllers may also be used to maintain the cavity and its gases within a targeted temperature range.

The optical cavity has at least one, highly-reflective optical mirror mounted on each side of the optical cavity, which cause the laser beam to reflect back and forth within the optical cavity to increase the effective path length of the beam. Preferably, the mirrors have a reflectivity of at least approximately 0.9995 and are coated for MIR wavelength light. The mirrors are preferably fixed to adjustable mounts, which orientation is adjustable by the controller. In some embodiments, at least one mirror may be mounted on piezoelectric (PZT) actuators or stacks as a means to maintain cavity alignment or to vary the exact length of the optical cavity.

In a preferred embodiment, the laser produces a beam having a wavelength in mid-infrared (MIR) spectrum, preferably at a wavelength of approximately 4.6 microns. The laser may be, for example a quantum cascade laser, preferably, a thermo-electrically cooled laser or preferably a continuous wave laser.

### BRIEF DESCRIPTION OF THE DRAWINGS

FIG. 1 is a schematic illustration of a carbon monoxide (CO) breath analyzing apparatus in accordance with an embodiment of the invention;

FIG. 2 is a block diagram of the breath sampling unit of the apparatus shown in FIG. 1;

FIG. 3 is a schematic illustration of the breath sampling unit of the apparatus show in FIG. 1;

FIGS. 4A-B illustrate example plot of the absorption coefficient (units of cm-1) for a concentration of 1 ppm of CO showing the P and R branches of the fundamental CO band, according to one embodiment.

FIG. 5 illustrates an example plot of the absorption coefficient versus frequency for a concentration of 1 ppb of CO and 1% water concentration, according to one embodiment.

FIGS. 6a, 6b and 6C illustrate example plots of the absorption coefficient versus frequency for a concentration of 100 ppb of CO and 1% water concentration, according to one embodiment.

FIG. 7 illustrates an example CO breath analyzer system 900 that can control the laser and cavity frequencies, according to one embodiment.

### DETAILED DESCRIPTION OF PREFERRED EMBODIMENTS

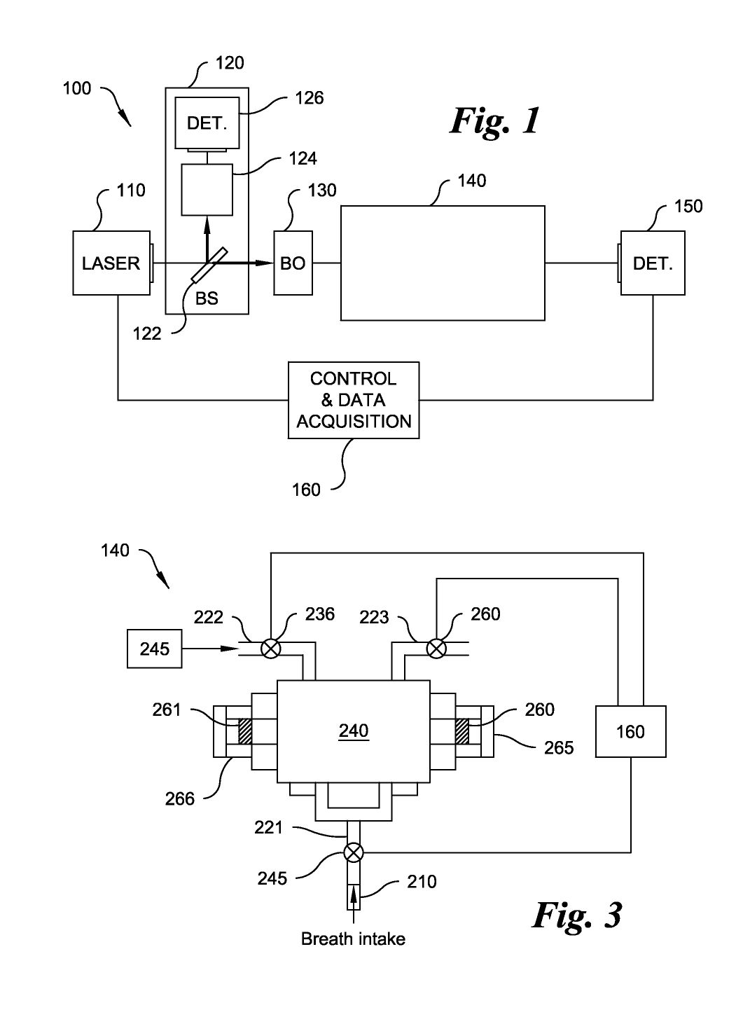

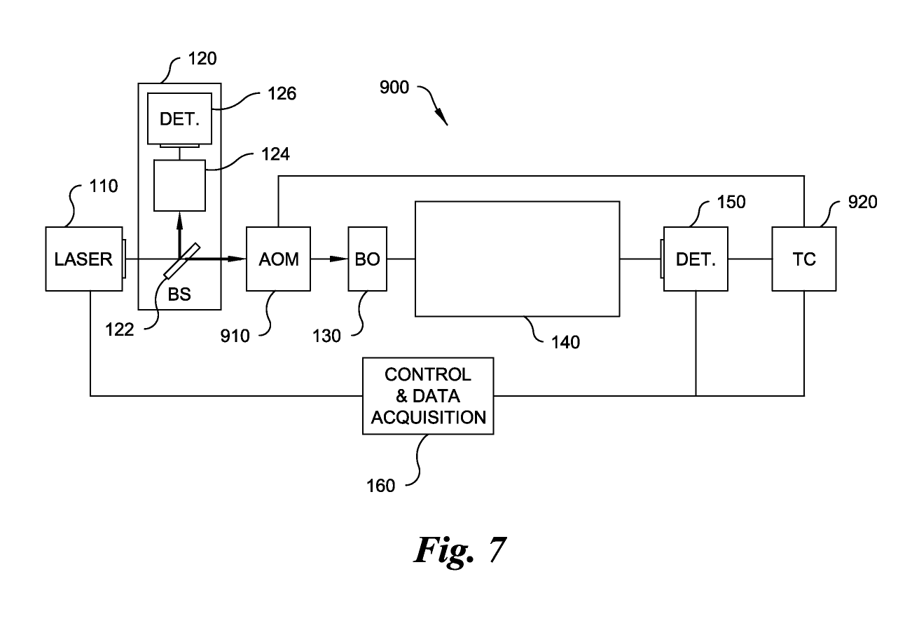

A carbon monoxide (CO) breath analyzing apparatus in accordance with an embodiment of the invention is shown in FIGS. 1-7 and is designated generally be reference numeral 100. The apparatus can measure CO at sensitivity levels as low as parts per billion (ppb). The apparatus 100 generally includes a laser 110, laser beam frequency measurement optics 120, laser beam shaping optics 130, breath sampling unit 140, a photo detector 150 and a control and data acquisition system 160. The laser 110 provides illumination for the system 100. The laser beam frequency measurement optics 120 provide precise frequency measurements of the laser beam (or simply beam). The beam shaping optics 130 condition the beam to the appropriate mode diameter and curvature. As shown in FIG. 7, a modulator (e.g. acousto-optic modulator) or other device may be used to extinguish the beam prior to cavity injection. The breath sampling unit 140 collects the breath sample and contains it within in a controlled-atmosphere, optical cavity 240 in which the beam traverses and decays as it passes through the breath sample. The optical cavity 240 has highly-reflective mirrors on each side, which cause the beam to reflect back and forth within the optical cavity 240 to increase the effective path length of the beam. The photo detector 150 measures decay (“ring-down”) of the beam as it repeatedly traverses the breath sample within the optical cavity 240. The control and data acquisition system 160 calculates the CO concentration of the breath sample in real time based on the decay values received from the photo detector 150.

The system 100 measures CO concentration utilizing absorption spectroscopy. Absorption spectroscopy measures the amount of light absorbed by the CO and can correlate this to the concentration of CO within the sample. The use of the cavity 210 for absorption spectroscopy is known as cavity enhanced absorption spectroscopy (CEAS). In a preferred embodiment, the apparatus performs cavity-enhanced ring-down spectroscopy on the sample. However, various other versions of CEAS can be performed, including integrated cavity output spectroscopy (ICOS), phase-shift cavity ring-down spectroscopy (PS-CRDS), continuous wave cavity enhanced absorption spectrometry (cw-CEAS), noise-immune cavity-enhanced optical-heterodyne molecular spectroscopy (NICE-OHMS), and related techniques that use optical cavities to achieve high detection sensitivity.

In one embodiment, the laser 110 operates in the mid-infrared (MIR) spectrum. Operating the laser 110 in the MIR spectrum enables the system to detect CO at the fundamental vibrational band of CO. Detection of CO at its fundamental vibrational band allows higher detection limits due to the higher absorption strengths of these transitions. According to one embodiment, the laser will operate at approximately 4.6 microns, corresponding to the P- and R-branches of the fundamental band. A particularly attractive absorption line within this region is the R6 absorption line of CO. The R6 absorption line of CO provides CO detection without interference from other gas species and with limited interference from water.

The laser 110 may be a quantum cascade laser (QCL). QCLs achieve gain via the transitions of electrons between two sub-bands in the conduction band of a coupled quantum well structure. The output wavelength is determined by the thickness of the active region and is independent of the band gap allowing access to the strong fundamental transitions of many molecules (including CO) which tend to be located in the MIR spectrum. The laser 110 may also be a continuous wave QCL, an external cavity QCL, a thermo-electrically cooled QCL, a commercially available QCL, or some combination thereof. In a preferred embodiment, the laser 110 is an external cavity, thermo-electrically cooled, continuous wave QCL. A thermo-electrically cooled laser does not require the cumbersome cooling systems (e.g., liquid nitrogen cooling) associated with some other lasers. Elimination of such cumbersome cooling system reduces the size of the apparatus 100 and increases its portability. The laser 110 may have power output in approximately the 3 to 50 mW range. The laser 110 may have a linewidth of approximately 3-50 MHz. The laser 110 may provide continuous mode hop free tuning in the MIR wavelength region. The linewidth may be narrow compared to the absorption linewidth, and the combination of the linewidth and power may be sufficient for injecting enough cavity power to have high signal-to-noise detection.

The laser beam frequency measurement optics 120 may form an optical reference leg for precise laser beam frequency measurement. In the embodiment show in FIG. 1, the frequency measurement optics 120 includes a beam splitter 122, an etalon 124, and an optical detector 126. The beam splitter 122 may split the beam so that the laser beam is provided to the laser beam shaping optics 130 and the etalon 124. The etalon 124 may be used to remove resonances from the beam and the photo detector 126 may be used to measure wavelength of the beam. It may be possible to measure the beam frequency with other components or methods. The frequency measurement optics 120 should preferably be appropriate for light in the MIR spectrum. The frequency measurement optics 120 may be made from zinc-selenium (ZnSe) or other infrared components.

The beam shaping optics 130 may condition and deliver the beam with low loss optical components including prisms, lenses, and/or irises to achieve the appropriate mode diameter and curvature. The beam shaping optics 130 may be appropriate for light in the MIR spectrum. The beam shaping optics may be made from ZnSe or other infrared components.

The breath sample unit 140 collects the breath sample and contains it within a controlled atmosphere in the optical cavity 240 wherein absorption spectroscopy, more particularly, cavity-ring down spectroscopy (CRDS), is preferably performed. The use of the reflective mirrors within the optical cavity 240 dramatically increases effective path lengths and detection sensitivities. The CRDS is the measurement of the decay (“ring-down”) of laser light within a high finesse optical cavity containing an absorbing sample (which in the system 100 is CO gas).

The photo-detector 150 measures the light exiting the optical cavity 140. The photo-detector 150 may be, for example, a Mercury Cadmium Telluride (HgCdTe) based detector. The signals measured by the detector 150 are transmitted to the control and data acquisition system 160, which calculates the CO concentration based on the decay (“ring-down”) of the beam. The control and data acquisition system 160 may compare the measurements to measurements for known CO concentrations. The control and data acquisition system 160 may store and display the data.

The control and data acquisition system 160 preferably controls the operation of the system 100. The control and data acquisition system 160 may be a computer or a processor. The control and data acquisition system 160 may include machine readable storage medium for storing instructions, which when executed by a machine (processor, computer) causes the machine to control the apparatus 100 including, for example, storing the received data, graphing/charting the data, calculating the CO concentration, graphing/charting the CO concentrations and/or adjusting the laser 110, as well as the modulator and PZT if they are used.

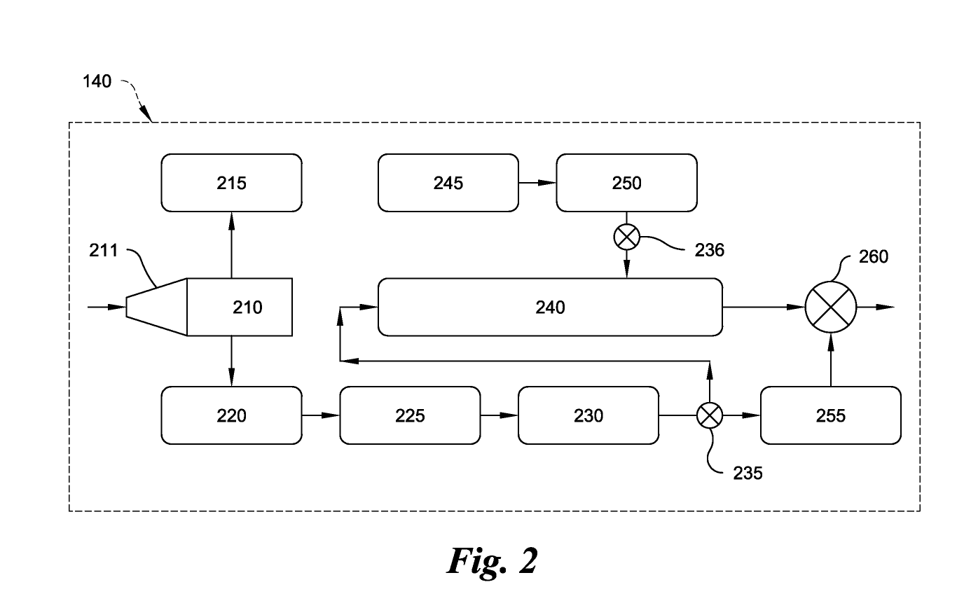

The breath sampling unit 140 is functionally illustrated in FIG. 2. The unit 140 includes a mouthpiece 210, an optional spirometer 215, a water and gas adsorption unit 220, a mass flow controlling unit 225, a pressure monitoring unit 230, a control valve 235, an optical cavity 240, a buffer gas supply 245, a buffer gas control unit 250, a cavity pressure control mechanism 255, and a control valve 260. The mouthpiece 210 preferably includes a disposable, filtered tip that is exchanged each time the apparatus 100 is used to test a different patient. In a preferred embodiment, the optional spirometer 215 measures the amount of air and the rate of air that is exhaled by the patient into the mouth piece 210 for the purpose of primary diagnosis. The breath sampling unit 140 would function equally effectively without the spirometer. The water and gas adsorption unit 220 absorbs the water and gas, especially carbon dioxide and water vapor from the exhaled breath of the patient. The control unit 225, pressure monitoring unit and valve 235 measure and control the flow of the breath sample into the optical cavity. These functions may be controlled by a single integrated unit or separate units. The optical cavity 240 encapsulates the breath sample and provides an enhanced cavity (described below) in which to perform ring down spectroscopy on the breath sample.

The optical cavity 240 is preferably purged with a buffer gas, such as nitrogen, prior to introduction of the breath sample into the optical cavity 240. The buffer gas source 445 is arranged in fluid communication with the optical cavity 240. The buffer gas control unit 250, which is preferably located intermediate the gas source 245 and the optical cavity 240, monitors and controls the pressure and flow rate of the buffer gas into the optical cavity 240. The volume and pressure of gas within the cavity is controlled by a valve 260 and control unit 255. As the breath sample is introduced into optical cavity 240, the valve 260 is opened, which allows the purge gas to be replaced by the breath sample. In a preferred embodiment, each function of the breath sampling unit is electronically controlled by a single controller, such as the control and data acquisition system 160.

The sampling unit 140 is schematically illustrated in FIG. 3. The sampling unit includes an optical cavity 240, an purge gas input tube 222 with a control valve 236, an vent tube 223 with a control valve 260, a breath intake tube 221 with a mouth piece 210 and a control valve 235, highly reflective mirrors 260, 261, which are fixed to adjustable mirror mounts 265, 266 with a piezoelectric (PZT) transducer mounts (not shown). The purge gas input and vent tubes 222, 223 extend from external sources to the optical cavity 210. The breath intake flow tube 221 extends from the external mouth piece 210 to the optical cavity 240.

The optical cavity 210 should be as small as possible without adversely affecting the sensitivity of the apparatus. For example, the optical cavity 240 may have a length of approximately 20-50 cm. The optical cavity 240 should also preferably have a high stability factor (g-parameter). The mirrors 260, 261 should be highly reflective and may be coated for MIR wavelengths, i.e., approximately 4.6 microns. For example, the mirrors 260, 261 may have reflectivities of greater than approximately 0.9995. In one embodiment, the mirrors 260, 261 have a radius of curvature of approximately 1 m. The mirror mounts 265, 266 position the mirrors 260, 261 in the appropriate location and at the appropriate angle so that the beam reflects back and forth many times. In a preferred embodiment, the mirrors 260, 261 are positioned so that the laser beam reflects approximately 10⁴ times as would be the case with reflectivity of 0.9999. In a preferred embodiment, the mirror mounts 265, 266 can be electronically adjusted, e.g. via PZTs, by the control and data acquisition system 160 or a separate controller.

In a preferred embodiment, the apparatus performs CRDS on the breath sample contained within the optical cavity. The control and data acquisition unit is programmed to calculate the CO concentration based on the following calculations. However, it should be appreciated to those of ordinary skill in the art that different CEAS techniques, such as ICOS, could be substituted for CRDS without departing from the scope of the invention.

CRDS is the measurement of the decay (“ring-down”) of the laser beam within the optical cavity as the laser beam repeatedly traverses the breath sample, which contains particles of CO gas. Under appropriate conditions, the ring-down signal S(t,ν) decays exponentially versus time (t) as

$${Abs}_{Eff} ( v ) ≡ {l}_{abs} {k}_{Eff} ( v ) = \frac{l}{c} [ \frac{1}{τ ( v )} - \frac{1}{{τ}_{0}} ]$$

where ν is the laser frequency, τ is the 1/e time of the decay (termed the ring-down time), c is the speed of light, l is the cavity length, k_{eff} (ν) is the effective absorption coefficient (including laser broadening), l_{abs} is the absorber path length (=1 if the sample fills the cavity), and 1−R is the effective mirror loss (including scattering and all cavity losses). In practice, the measured ring-down signal S(t, ν) is fitted with an exponential, and the ring-down time τ is extracted. Combining τ with the “empty cavity ring-down time”, τ₀ (measured by detuning the laser from the sample absorption and/or fitting the baseline) allows determination of the effective absorbance Abs_{Eff}(ν), which is the fractional amount of light absorbed per pass through the cavity, and equals the product of the absorber path length and effective absorption coefficient, k_{Eff}, such that

$$S ( t , v ) = {S}_{0} exp [ - t / τ ( v ) ]$$

$$1 / τ ( v ) = \frac{c}{l} [ {k}_{Eff} ( v ) {l}_{abs} + ( 1 - R ) ]$$

In a preferred embodiment, the laser frequency is scanned across the absorption line and the frequency-integrated spectrum (i.e., the line area) is measured. The line area measured in this way can be readily converted to the path-integrated concentration of the absorbing species if the temperature and relevant spectroscopic constants are known.

The CO concentration will be approximately spatially uniform within the cavity so a spatially averaged concentration will be determined by dividing the path-integrated concentration by the absorber path length. Spectral simulations are performed in order to identify optimum line(s) for measurement, study system sensitivity and consider possible spectral interferences. The sensitivity of a given CRDS setup, in terms of the minimum measurable absorbance, is given as:

Abs_{Min}=(1−R)(Δτ/τ)_{Min}

where (Δτ/τ)_{Min} is the minimum experimentally measurable fraction change in ring-down time, and 1−R is the mirror loss. In the preferred embodiment, using a laser in the MIR range of approximately 4.5-4.6 μm enables detection of CO at its fundamental vibrational band. In this spectral region, mirrors having a reflective factor of approximately R=0.9998 are available and may be used such that the mirror loss 1−R is approximately 0.0002. Using a continuous wave laser, and a 10 s measurement times yields a conservatively estimate that for 10 s measurement times we will have a fractional precision (sensitivity) of (Δτ/τ)_{Min} less than or equal approximately 10⁻³. Accordingly, the minimum detectable absorbance Absₘᵢₙ would be (0.0002)(0.001) or approximately 2×10⁻⁷ (or 200 ppb optical absorbance). Other values of reflectivity will give correspondingly different sensitivities.

If the cavity has an absorber path length of approximately 20 cm this would result in a detection limit (spatially averaged concentration) of approximately 10⁻⁸ cm⁻¹. The detection limit scales approximately as square-root of the measurement time so that, for example, a 1 s measurement time would degrade the detection limit by a factor of approximately 3. The minimum detectable values also correspond to the system precision. Owing to the directly quantitative nature of CRDS, the accuracy is estimated to be better than 1 part in 300 (likely 1 part in 1000). The accuracy may be verified in calibration tests using premixed gas cylinders of known CO concentrations.

In a preferred embodiment, spectral modeling is used to convert the measurable optical absorbance to species concentration. Spectral simulations may be performed utilizing the high-resolution transmission molecular absorption database (HITRAN) tool for CO and H2O (including all bands and isotopes) in the spectral range of interest. The HITRAN is a compilation of spectroscopic parameters that a variety of computer codes use to predict and simulate the transmission and emission of light in the atmosphere. The spectral simulations for the CO detection system assume a pressure of approximately 1 atmosphere (atm) and a temperature of approximately 295 degrees Kelvin (K). Optimum pressure may be determined to slightly sub-atmospheric (e.g., in the range of 0.1-0.5 atm), but will not significantly degrade the apparatus' performance.

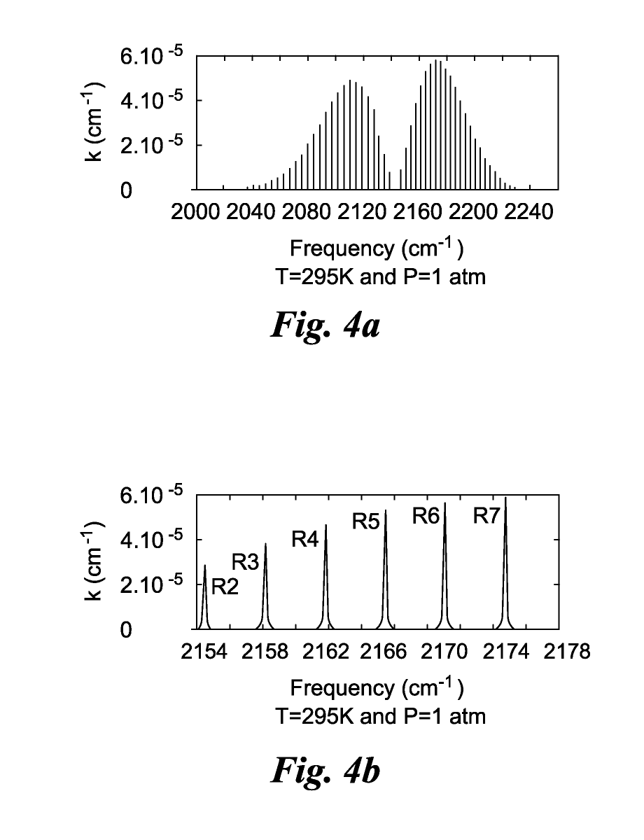

FIG. 4A illustrates an example plot of the absorption coefficient (units of cm⁻¹) for a concentration of 1 ppm of CO versus the frequency of the P and R branches in the fundamental CO band. FIG. 4B illustrates an example exploded plot showing only the concentration of R branches in close proximity to the R6 branch. As illustrated, the R6 branch has a peak absorption coefficient (k) of approximately 6×10⁻⁵ cm⁻¹. This corresponds to a detection limit of approximately 0.17 ppb (10⁻⁸ cm⁻¹/6×10⁻⁵ cm⁻¹)×1 ppm). Thus in the absence of interference, the apparatus may measure CO at less than 1 ppb concentrations.

For line selection, spectral interferences due to water may also be considered. The interference is limited to water because no other air species interferes in this region. If a relative humidity (RH) of approximately 50% is assumed this would correspond to a water concentration (molar) of approximately 1%.

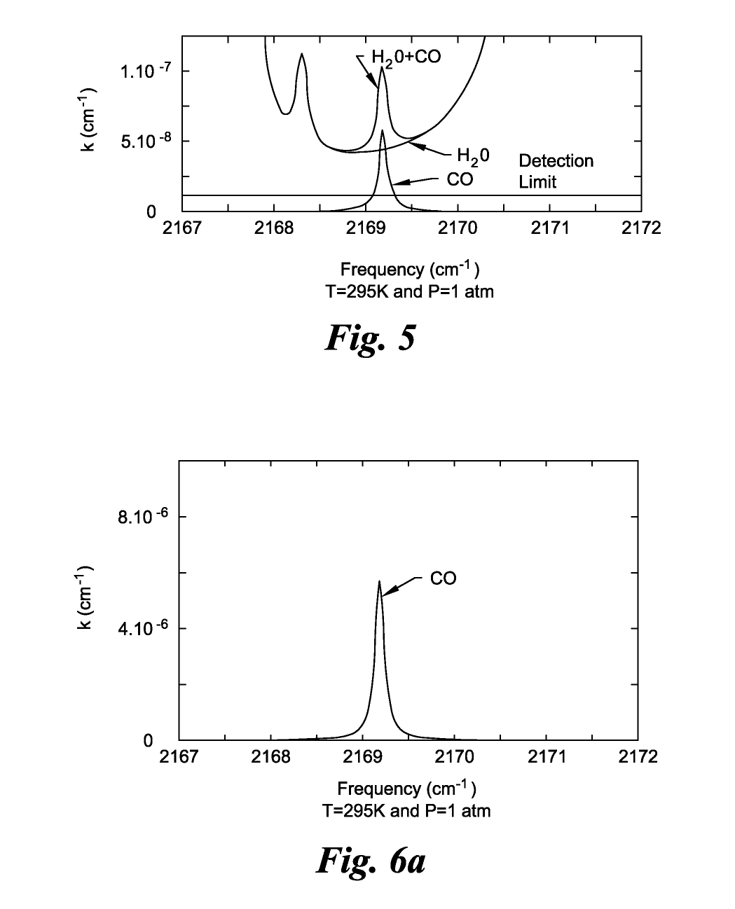

FIG. 5 illustrates an example plot of the absorption coefficient versus frequency for a concentration of 1 ppb of CO and 1% water concentration. The plot illustrates the concentration of CO, water and the combination of CO and water. As illustrated, for the R6 line the 1% water contributes an absorption from the wings of adjacent features equivalent to less than 1 ppb CO. The measured spectrum can be adjusted in order to subtract off the baseline such that the water will have negligible effect and the detection limit may be as illustrated. Alternatively, in the absence of baseline fitting and subtraction, the presence of 1% water is the limiting factor in the ability to detect CO yielding a detection limit of approximately 1 ppb CO.

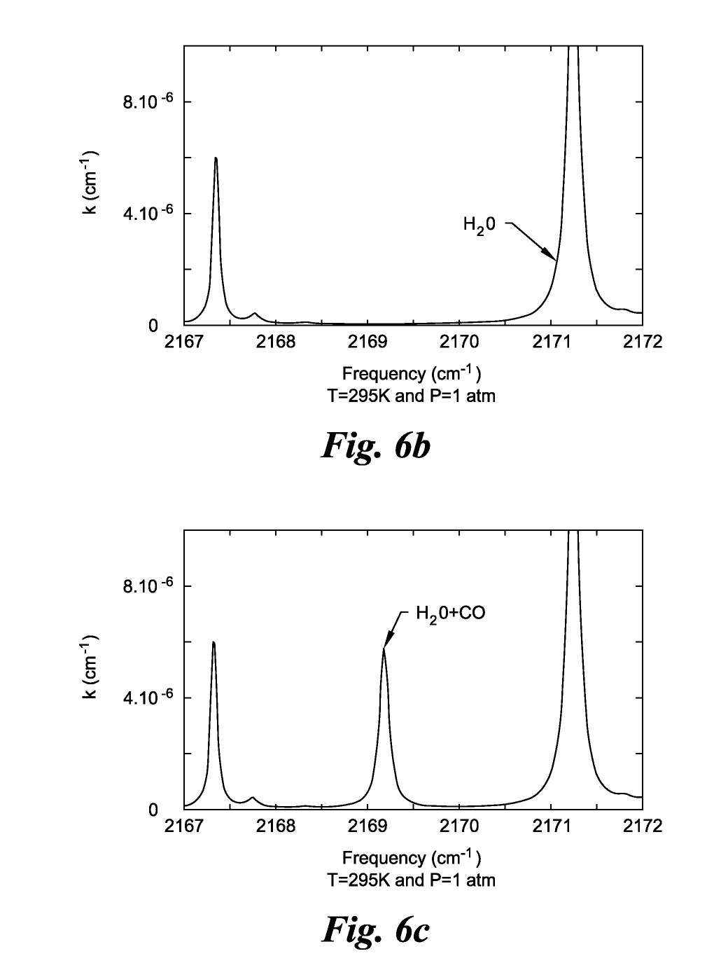

FIG. 6 illustrates an example plot of the absorption coefficient versus frequency for a concentration of 100 ppb of CO and 1% water concentration. As illustrated, baseline fitting and subtraction is not included so the effect of the water is to reduce the measurement precision to approximately 1 ppb and to the accuracy to approximately 1%.

The system may be calibrated for accuracy and precision by using calibrated gas samples with different CO concentrations (ppm and ppb levels) and water concentrations to simulate interferences. CRDS provides a favorable combination of high detection sensitivity and directly quantitative measurements. Use of a commercial QCL system will allow a compact and rugged sensor. For 10 second measurement times, a conservative estimate a CO detection is less than approximately 1 ppb and accuracy better than approximately 1 part in 300. These sensitivities are superior to those available from existing commercial CO sensors. In addition to providing increased sensitivity, the sensor apparatus of the present invention may be robust, compact, and economically priced.

In continuous wave CRDS systems, the frequency of the laser and the cavity should be controlled to enable coupling of the narrow band laser light into the optical cavity since the cavity mode spacing exceeds the laser linewidth. This may be achieved by scanning the laser, e.g. with current or temperature modulation, and/or by scanning the cavity length, e.g. with the PZT.

FIG. 9 illustrates an example CO breath analyzer system 900 that can control the laser and cavity frequencies. The system 900 is similar to that discussed with regard to FIG. 1 and like parts are identified with like reference numbers. The system includes an acoustic optic modulator (AOM) 910 and a threshold circuit 920. The AOM 910 may be used to modulate the beam. The threshold circuit 920 may be used to determine when the frequencies of the beam and the cavity are overlapping.

According to one embodiment, the wavelength of the beam from the laser 110 will be continuously scanned by the laser beam frequency measurement optics 120 (optical reference leg) while the cavity 140 will not be actively scanned. The threshold circuit 920 may monitor for overlap of the laser frequency with cavity transmission peaks. The overlap may be detected when the detector 150 measures an increasing light signal. When the overlap is detected, the TC 920 may trigger the AOM 910 to extinguish the light delivered to the cavity 140. The AOM 910 may turn off if the AOM 910 has already presented the first order beam to the cavity 140.

Subsequent to the extinction, light within the cavity 140 will decay yielding an exponential ring-down signal, which is measured by the detector 150 and converted to CO concentration by the control and data acquisition system (e.g., computer) 160. Controlling the frequencies of the cavity 140 and beam in this fashion is simple but may suffer from insufficient wavelength resolution since it neglects passive cavity drift. The wavelength-spacing of points in the measured spectrum will be approximately equal to the cavity Free Spectral Range (FSR) that is approximately 300 MHz, which may not be sufficiently small compared to the width of the absorption line (Full-Width-Half-Maximum) if the line is several GHz depending on cell pressure. The resulting spectrum will be fit to determine the total absorption (e.g., the wavelength integrated absorption). The fitting of the spectrum to absorption may include numerical integration, non-linear Voigt fitting (e.g., least-squares), and comparison against “look up table” fits from modeling. Different methods for baseline subtraction (and effect of the water interference) may also be used.

According to one embodiment, the cavity 140 may be brought into resonance with the laser 110 by scanning the position of the rear cavity-mirror with a piezoelectric (PZT) stack (not illustrated). The detector 150 may still be used to monitor coupling. This approach allows more tightly spaced points on the wavelength axis.

In order to precisely determine the wavelength axis, a simple reference leg 120 may be used. A small portion of the laser beam will be picked-off and passed through the etalon 124 (e.g., a zinc selenide etalon with FSR of approximately 2 GHz), and the etalon transmission peaks measured by the detector 126 may be used for precise calibration of the wavelength axis. The readout accuracy of the wavelength of the QC laser 110 (approximately 0.01 cm⁻¹) may be sufficient that a reference cell should is not needed. However, if a reference cell is needed it can be added without departing from the current scope.

A concentration measurement based on a single line requires knowledge of temperature, which may be independently obtained with thermocouples (not illustrated). The temperature may also be determined by other sensors or spectroscopically from the strengths of absorption lines. The laser 110 may be repetitively scanned over the targeted absorption line(s) and the signals averaged to increase measurement signal to noise.

It should be noted that the disclosure focused on a QCL due to advantages noted including the laser being commercially available, easy to use and being electro thermally cooled but is not limited thereto. Rather, other lasers could be used for providing a laser beam in the MIR spectrum, such as lead-salt lasers, without departing from the current scope. Furthermore, the disclosure focused on CRDS due to the noted advantages including providing a favorable combination of high detection sensitivity and directly quantitative measurements but is not limited thereto. Rather other CEAS methods such as integrated cavity output spectroscopy (ICOS) could be used without departing from the current scope.

Although the disclosure has been illustrated by reference to specific embodiments, it will be apparent that the disclosure is not limited thereto as various changes and modifications may be made thereto without departing from the scope. Reference to “one embodiment” or “an embodiment” means that a particular feature, structure or characteristic described therein is included in at least one embodiment. Thus, the appearances of the phrase “in one embodiment” or “in an embodiment” appearing in various places throughout the specification are not necessarily all referring to the same embodiment.

The various embodiments are intended to be protected broadly within the spirit and scope of the appended claims.

1. A system for non-invasively measuring carbon monoxide (CO) traces in a patient's exhaled breath, the system comprising:
a) a laser to provide a light having a wavelength in mid-infrared (MIR) spectrum;
b) an optical cavity constructed and arranged to perform cavity enhanced absorption spectroscopy (CEAS) including highly reflective mirrors mounted to each side of the optical cavity to cause the light received from the laser to bounce back and forth within the optical cavity to increase effective path length of the light;
c) a breath intake tube connected to the optical cavity for collecting a sample of the patient's exhaled breath and transferring it to the optical cavity;
d) a photo detector to measure parameters about the light exiting the optical cavity; and,
e) a controller to control operation of the system and determine the concentration of CO in the breath sample based on measurements from the photo detector;
wherein said system measures the CO concentration at sensitivity levels at least as low as parts per billion.

2. The system of claim 1, wherein the laser is a quantum cascade laser.

3. The system of claim 2, wherein the quantum cascade laser is a thermo-electrically cooled laser.

4. The system of claim 2, wherein the quantum cascade laser is a continuous wave laser.

5. The system of claim 4, wherein the laser is an external cavity laser.

6. The system of claim 1, wherein the laser operates at a wavelength of approximately 4.6 microns.

7. The system of claim 1, wherein the optical cavity is constructed and arranged to perform cavity-ring down spectroscopy (CRDS).

8. The system of claim 1, wherein the optical cavity is constructed and arranged to perform integrated cavity output spectroscopy (ICOS),

9. The system of claim 1, wherein the highly reflective mirrors have a reflectivity of about 0.9995 to about 0.9999 and are coated for MIR wavelength light.

10. The system of claim 1, wherein the optical cavity includes controller adjustable mounts to hold the highly reflective mirrors and to adjust configuration of the highly reflective mirrors when instructed to do so by the controller.

11. The system of claim 1, wherein said breath intake tube includes a flow meter to measure flow and an adjustable valve to control flow and volume of the patient's exhaled breath provided to the optical cavity.

12. The system of claim 1, further comprising means for purging said optical cavity with a buffer gas.

13. The system of claim 12, wherein said purging means includes a gas source, an inlet tube connecting said gas source to said optical cavity, an outlet tube connecting the optical chamber to the atmosphere, and means to control the flow of the purging gas.

14. The system of claim 1, wherein said controller includes a processor and a controller-readable storage medium storing controller executable instructions that when executed by the controller cause the controller to control operation of the system and determine the concentration of CO.

15. A method for non-invasively measuring carbon monoxide (CO) traces at parts per billion (ppb) levels in a patient's exhaled breath, comprising the steps of:
a) collecting a sample of a patient's exhaled breath in an optical cavity via a breath intake tube connected to the optical cavity;
b) performing cavity enhanced absorption spectroscopy (CEAS) on the breath sample by:
i) illuminating the breath sample in the optical cavity with a laser beam having a wavelength in the mid-infrared (MIR) spectrum;
ii) reflecting the laser beam back and forth within the optical cavity using highly reflective mirrors mounted to each side of the optical cavity to increase the effective path length of the laser beam passing through the breath sample;
iii) measuring decay of the laser beam exiting the optical cavity; and
c) determining the CO concentration in the breath sample at sensitivity levels at least as low as parts per billion of CO based on the measured decay.

16. The method of claim 15, wherein CEAS is performed using cavity ring-down spectroscopy (CRDS).

17. The method of claim 15, wherein CEAS is performed using integrated cavity output spectroscopy (ICOS).

18. The method of claim 15, further comprising the steps of:
d) measuring flow of the breath sample using a flow meter; and
e) controlling the flow and volume of the breath sample to the optical cavity.

19. The method of claim 15, further comprising the steps of:
f) initially purging the optical cavity with a purge gas prior to collecting the breath sample.

20. The method of claim 15, wherein the breath sample is illuminated with a laser beam from a continuous wave thermo-electrically cooled quantum cascade laser.

21. The method of claim 15, wherein the breath sample is illuminated with a laser from an external cavity laser.

22. The method of claim 13, wherein the breath sample is illuminated with a laser beam having a wavelength of approximately 4.6 microns.

23. A system for non-invasively measuring carbon monoxide (CO) traces at parts per billion (ppb) levels in a patient's exhaled breath, the system comprising:
a) a continuous-wave, thermo-electrically cooled quantum cascade laser to provide a laser beam having a wavelength of approximately 4.6 microns;
b) an optical cavity to perform cavity-ring down spectroscopy (CRDS) including a highly reflective mirror mounted to each side of the optical cavity to cause the laser beam to reflect back and forth within the optical cavity to increase the effective path length of the laser beam;
c) a breath intake tube connected to the optical cavity for collecting a sample of the patient's breath and conveying it to the optical cavity, said breath intake tube including a mouthpiece, a flow meter, and an adjustable flow valve;
d) means for purging said optical cavity comprising a gas source, an inlet tube connecting said gas source to said optical cavity, an outlet tube connecting the optical chamber to the atmosphere, and means to control the flow of the purging gas to the optical cavity;
e) a photo detector to measure decay of the laser beam exiting the optical cavity;
f) a processor; and,
g) a processor-readable storage medium storing processor executable instructions that enable the processor to determine the concentration of CO based on the decay.

24. The system of claim 23, further comprising beam shaping optics to condition the laser beam to achieve appropriate mode diameter and curvature for delivery to the optical cavity.
